(19) United States
(12) Patent Application Publication
(10) Pub. No.: US 2017/0056614 A1
(43) Pub. Date: Mar. 2, 2017

(54) APPARATUS, SYSTEM, AND METHOD FOR INHALING AROMAS

(71) Applicants: Troy Thuet, Midvale, UT (US); J K Kim, Midvale, UT (US); Eui Seok Kim, Goyang-si (KR)

(72) Inventors: Troy Thuet, Midvale, UT (US); J K Kim, Midvale, UT (US); Eui Seok Kim, Goyang-si (KR)

(21) Appl. No.: 15/246,378

(22) Filed: Aug. 24, 2016

Related U.S. Application Data
(60) Provisional application No. 62/209,175, filed on Aug. 24, 2015.

(51) Int. Cl. A61M 21/02; A61M 15/06; A61M 15/00

(52) U.S. Cl. CPC A61M 21/02; A61M 15/0025; A61M 15/06; A61M 2205/75; A61M 2021/0016

(57) ABSTRACT

An apparatus, system, and method are disclosed for inhaling an aroma. The apparatus to inhale an aroma includes a cylindrical tube, an aroma dispensing member, and a filter. The cylindrical tube is elongated in a lengthwise direction, extending between a proximal end and a distal end. The proximal end of the cylindrical tube is in fluid communication with an exterior of the cylindrical tube. The distal end of the cylindrical tube is also in fluid communication with an exterior of the cylindrical tube. The aroma dispensing member is positionable within an interior of the cylindrical tube between the proximal end of the cylindrical tube and the distal end of the cylindrical tube. The filter is also positionable within the interior of the cylindrical tube between the aroma dispensing member and the proximal end of the cylindrical tube.

### CROSS-REFERENCES TO RELATED APPLICATIONS

This application claims the benefit of U.S. Provisional Patent Application No. 62/209,175 entitled “APPARATUS, SYSTEM, AND METHOD FOR INHALING AROMAS” and filed on Aug. 24, 2015 for Thuet et al., which is incorporated herein by reference.

### FIELD OF THE INVENTION

This invention relates to aroma therapy and more particularly relates to an apparatus for inhaling aromas.

### BACKGROUND

Smoking cessation or quitting tobacco is the process of discontinuing tobacco use. Tobacco contains nicotine, which is addictive, making the process of quitting often very prolonged and difficult. Quitting tobacco often leads to symptoms of nicotine withdrawal such as craving, anxiety and irritability, depression, and weight gain.

Many different strategies can be used for smoking cessation, including quitting without assistance (“cold turkey” or cut down then quit) and the use of medications such as nicotine replacement therapy (NRT) or varenicline. Quitting without assistance has proven very difficult and studies have shown that only 3% to 6% of user's who attempt to go “cold turkey” are successful.

The use of medications such as NRT or varenicline has been shown to be more effective in quitting tobacco. For example, a study of NRT has shown that user's using this form of therapy increases the chances of quitting tobacco by 50 to 70%. Similarly, user's who use varenicline to aid in tobacco cessation saw a 23% increase in continuous abstinence after one year.

However, the use of these substances are not without their own problems. For example, NRT's contain nicotine and are therefore addictive in and of themselves. Varenicline, on the other hand, has been demonstrated to increase a user's risk of serious adverse cardiovascular events. Accordingly, an alternative to these forms of therapy for tobacco cessation is desirable.

The present invention has been developed in response to the present state of the art, and in particular, in response to the problems and needs in the art that have not yet been fully solved by currently available smoking cessation strategies and devices.

### SUMMARY

Aromatherapy uses plant materials and aromatic plant oils, including essential oils, and other aromatic compounds for the purpose of altering one's mood, cognitive, psychological or physical wellbeing. Using such a therapy helps in treating symptoms of nicotine withdrawal. However, currently available aromatherapy dispensers include aerial diffusers that dispense aroma's to the entire environment. Individuals who do not wish to be subjected to the aroma must leave the area in order to avoid the aroma. Furthermore, an aerial diffuser does not allow a user who is trying to quit smoking to manually manipulate the diffuser in a manner similar to the way that a smoker handles a cigarette.

From the foregoing discussion, it should be apparent that a need exists for an apparatus, system, and method that delivers an aroma to an individual without subjecting others to the aroma. Beneficially, such an apparatus, system, and method would be dimensioned similar to the dimensions of a conventional cigarette. Accordingly, the present invention has been developed to provide an apparatus, system, and method for inhaling aromas that overcomes many or all of the above-discussed shortcomings in the art.

The apparatus to inhale an aroma, in one embodiment, includes a cylindrical tube, an aroma dispensing member, and a filter. The cylindrical tube is elongated in a lengthwise direction, extending between a proximal end and a distal end. The proximal end of the cylindrical tube is in fluid communication with an exterior of the cylindrical tube. The distal end of the cylindrical tube is also in fluid communication with an exterior of the cylindrical tube. The aroma dispensing member is positionable within an interior of the cylindrical tube between the proximal end of the cylindrical tube and the distal end of the cylindrical tube. The filter is also positionable within the interior of the cylindrical tube between the aroma dispensing member and the proximal end of the cylindrical tube.

In certain embodiments, the distal end of the cylindrical tube includes a barrier having at least one orifice extending from the interior of the cylindrical tube to the exterior of the cylindrical tube. In such an embodiment, the apparatus includes a stopper configured to limit fluid communication between the interior of the cylindrical tube and the exterior of the cylindrical tube through the distal end of the cylindrical tube and thereby selectively limit the dispensing of aromas through the distal end of the cylindrical tube. The stopper, in one embodiment, is a plug positionable within the at least one orifice. In other embodiments, the stopper is a cap positionable about the distal end of the cylindrical tube to selectively limit the dispensing of aromas through the distal end of the cylindrical tube.

The apparatus, in certain embodiments, also includes a mouthpiece coupleable to the proximal end of the cylindrical tube. The mouthpiece includes a passageway leading from interior of the cylindrical tube to the exterior of the cylindrical tube. In such an embodiment, a user may inhale the aroma by inhaling through the mouth piece. In an exemplary embodiment, the apparatus includes a cover positionable about the mouth piece. The cover is configured to limit fluid communication between the interior of the cylindrical tube and the exterior of the cylindrical tube through the proximal end of the cylindrical tube and thereby selectively limit the dispensing of aromas through the proximal end of the cylindrical tube.

An aroma dispensing substance disposed within the aroma dispensing member. In certain embodiments, the aroma dispensing substance has at least one therapeutic property. For example, in certain embodiments, the aroma dispensing substance is an appetite suppressing agent. In other embodiments, the aroma dispensing substance is a calming agent.

In certain embodiment, the aroma dispensing member is refillable with the aroma dispensing substance. For example, in certain embodiments, the aroma dispensing substance is an essential oil. In such an embodiment, once the aroma dispensing member has been depleted of the essential oil to the point where the essential oil is no longer therapeutically beneficial, the aroma dispensing member may be refilled with the aroma dispensing substance (i.e., the essential oil). In other embodiments, the aroma dispensing member is disposable and the apparatus is refilled by replacing the aroma dispensing member with a new aroma dispensing member.

The aroma dispensing member, in certain embodiments, includes a matrix of polyester fibers. In such an embodiment, the matrix of polyester fibers is disposed within a pliable sheath. The pliable sheath has an outer diameter smaller than an inner diameter of the cylindrical tube so that the aroma dispensing member can be positioned within the cylindrical tube.

The filter, in certain embodiments, is also made of a matrix of polyester fibers. In such embodiments, a pliable sheath encompasses the matrix of polyester fibers of the filter. The pliable sheath of the filter also has an outer diameter smaller than an inner diameter of the cylindrical tube so that the filter can be positioned within the cylindrical tube.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order that the advantages of the invention will be readily understood, a description of the subject matter will be rendered by reference to specific embodiments that are illustrated in the appended drawings. Understanding that these drawings depict only typical embodiments of the subject matter and are not therefore to be considered to be limiting of its scope, the subject matter will be described and explained with additional specificity and detail through the use of the accompanying drawings, in which:

FIG. 1 is an exploded perspective view illustrating one embodiment of an apparatus to inhale aromas in accordance with the present subject matter;

FIG. 2 is an exploded perspective view illustrating one embodiment of another apparatus to inhale aromas in accordance with the present subject matter; and

FIG. 3 is a perspective view illustrating one embodiment of the assembled apparatus to inhale aromas of FIG. 2 in accordance with the present subject matter.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided for a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Aromatherapy uses plant materials and aromatic plant oils, including essential oils, and other aromatic compounds for the purpose of altering one's mood, cognitive, psychological or physical wellbeing. Essential oils have been used medicinally in history. Aroma therapists, who specialize in the practice of aromatherapy, utilize blends of therapeutic essential oils to stimulate a desired response. The present invention is directed to an apparatus, system, and method to inhale an aroma comprised of an aroma dispensing substance (i.e., an essential oil) having at least one therapeutic property.

In an exemplary embodiment, the therapeutic property of the aroma dispensing substance includes a property aimed at treating one or more of the symptoms of nicotine withdrawal such as craving, anxiety and irritability, depression, and weight gain. Thus, the aroma dispensing substance may contain an antianxiety agent, a calming agent, an antidepressant, an appetite suppressing agent, or the like. In certain embodiments, the aroma dispensing substance may contain two or more of the above described agents. Of course, one of skill in the art will recognize that in certain embodiments, the aroma dispensing substance may contain a mixture of essential oils or other aroma containing substances that are aimed at treating individuals having a single cognitive, psychological or physical symptom. For example, in one embodiment, the aroma dispensing substance may contain a mixture of essential oils or other aroma containing substances that act as an appetite suppressant or a calming agent.

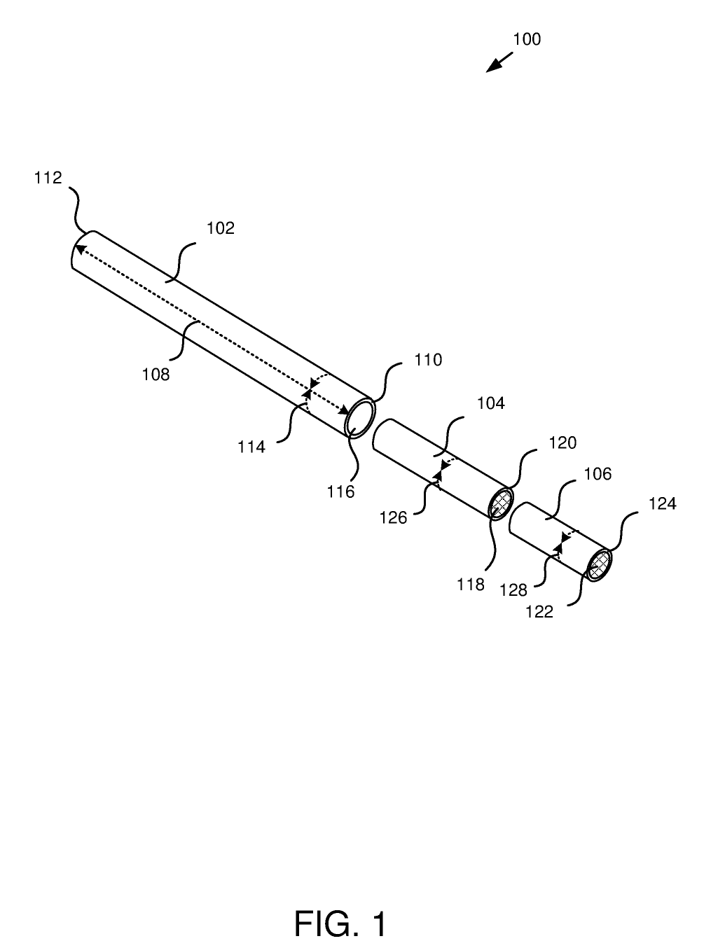

FIG. 1 is an exploded perspective view illustrating one embodiment of an apparatus 100 to inhale aromas in accordance with the present subject matter. The apparatus 100, in certain embodiments, includes a cylindrical tube 102, an aroma dispensing member 104, and a filter 106.

The cylindrical tube 102, in one embodiment, is elongated in a lengthwise direction 108 as indicated by line 108. The cylindrical tube 102 extends between a proximal end 110 and a distal end 112. In certain embodiments, the cylindrical tube 102 is sized and shaped substantially similar to a size and shape of a conventional cigarette. In such an embodiment, the outer diameter 114 of the cylindrical tube 102 is substantially similar to an outer diameter of a conventional cigarette. Similarly, the length 108 of the cylindrical tube 102 is substantially similar to an outer diameter of a conventional cigarette. By dimensioning the cylindrical tube 102 with dimensions substantially similar to the dimensions of a conventional cigarette, a user who is trying to quit smoking will get the same feel as handling a conventional cigarette.

In certain embodiments, the cylindrical tube 102 is made of a plastic or wooden material. In other embodiments, such as where durability may be an issue, the cylindrical tube 102 may be made of a metallic material. In an exemplary embodiment, the cylindrical tube 102 is made of aluminum for aluminum's strength to weight and durability characteristics. Aluminum, in one embodiment, may be preferred due to the fact that aluminum is easily cleaned. One of skill in the art will recognize that in other embodiments, the cylindrical tube 102 may be made of any material having a structural rigidity sufficient to maintain the shape of the cylindrical tube 102.

In one embodiment, the proximal end 110 of the cylindrical tube 102 is in fluid communication with an exterior of the cylindrical tube 102. The distal end 112 of the cylindrical tube 102 is also in fluid communication with an exterior of the cylindrical tube 102. Thus, in certain embodiments, air is free to flow from the exterior of the cylindrical tube 102 and through an interior 116 of the cylindrical tube 102. The free flow of air through interior 116 of the cylindrical tube 102 allows a user to inhale from the proximal end 110 of the cylindrical tube 102 causing air to flow into the distal end 112 of the cylindrical tube 102, through the interior 116 of the cylindrical tube 102, out the proximal end 110 of the cylindrical tube 102 and into the user's mouth or nose.

While the embodiment illustrated in FIG. 1, depicts the proximal end 110 and the distal end 112 as being substantially open, one of skill in the art will recognize that in other embodiments one or both of the proximal end 110 and the distal end 112 of the cylindrical tube 102 may include a barrier that substantially closes off the proximal end 110 and/or the distal end 112 of the cylindrical tube 102. In such an embodiment, the barrier(s) should include at least one orifice extending therethrough to allow for the free flow of air from the distal end distal end 112 of the cylindrical tube 102, through the interior 116 of the cylindrical tube 102 and out the proximal end 110 of the cylindrical tube 102.

The aroma dispensing member 104 is positionable within the interior 116 of the cylindrical tube 102 between the proximal end 110 of the cylindrical tube 102 and the distal end 112 of the cylindrical tube 102. In one embodiment, the aroma dispensing member is made of a matrix of polyester fibers 118 wrapped in a pliable sheath 120. The matrix of polyester fibers 118 act as an absorbent material for collecting and retaining an aroma dispensing substance such as an essential oil or a mixture of essential oils. As discussed above, in certain embodiments, the aroma dispensing substance has at least one therapeutic property (i.e., a calming agent, an antidepressant, an appetite suppressing agent, or the like.)

In certain embodiments, the aroma dispensing substance is absorbed within the aroma dispensing member 104 (i.e., within the matrix of polyester fibers 118) at the time of manufacturing the aroma dispensing member 104. In such an embodiment, to refill the apparatus with an aroma dispensing substance, the aroma dispensing member 104 is removed from the cylindrical tube 102 and replaced with a second aroma dispensing member 104. Thus, in certain embodiments, the aroma dispensing member 104 is prefilled with an aroma dispensing substance at the time of manufacture and the user simply replaces the aroma dispensing member 104 when the aroma of the aroma dispensing member 104 has been depleted. In other embodiments, the aroma dispensing substance may be applied to the aroma dispensing member 104 after the aroma dispensing member 104 has been manufactured. This allows a user to refill the aroma dispensing member 104 after the aroma dispensing substance has been depleted from the aroma dispensing member 104.

The pliable sheath 120, in one embodiment, is made of low density polyethylene. The pliable sheath 120 has an outer diameter 126 that is substantially smaller than the inner diameter of the cylindrical tube 102 such that the aroma dispensing member 104 fits within the interior 116 of the cylindrical tube 102. The pliable sheath 120 of the aroma dispensing member 104 acts to keep the matrix of polyester fibers 118 of the aroma dispensing member 104 contained within a column to easily insert the aroma dispensing member 104 into the interior 116 of the cylindrical tube 102.

The filter 106, in certain embodiments, is also positionable within the interior 116 of the cylindrical tube 102. The filter 106 is positioned between the aroma dispensing member 104 and the proximal end 110 of the cylindrical tube 102. Thus, in use, the aroma dispensing member 104 is positioned adjacent the distal end 112 of the cylindrical tube 102 and the filter 106 is positioned adjacent the proximal end 110 of the cylindrical tube 102. When a user inhales from the proximal end 110 of the cylindrical tube 102, air flows from the exterior of the cylindrical tube 102, into the distal end 112 of the cylindrical tube 102, through the aroma dispensing member 104 to pick up an aroma from the aroma dispensing substance, through the filter 106, through the proximal end 110 of the cylindrical tube 102 and into the user's mouth or nose.

The filter 106 acts to trap essential oils or other aromatic dispensing agents to keep the user from inhaling the essential oils or other aromatic dispensing agents themselves which may or may not contain harmful substances if ingested. In one embodiment, the filter 106 is also made of a matrix of polyester fibers 122 wrapped in a pliable sheath 124. The matrix of polyester fibers 122 traps any unwanted essential oils or other aromatic dispensing agents and the pliable sheath 124 of the filter 106 acts to keep the matrix of polyester fibers 122 of the filter 106 contained within a column to easily insert the filter 106 into the interior 116 of the cylindrical tube 102. The pliable sheath 124 of the filter 106 has an outer diameter 128 that is substantially smaller than the inner diameter of the cylindrical tube 102 such that the filter 106 fits within the interior 116 of the cylindrical tube 102.

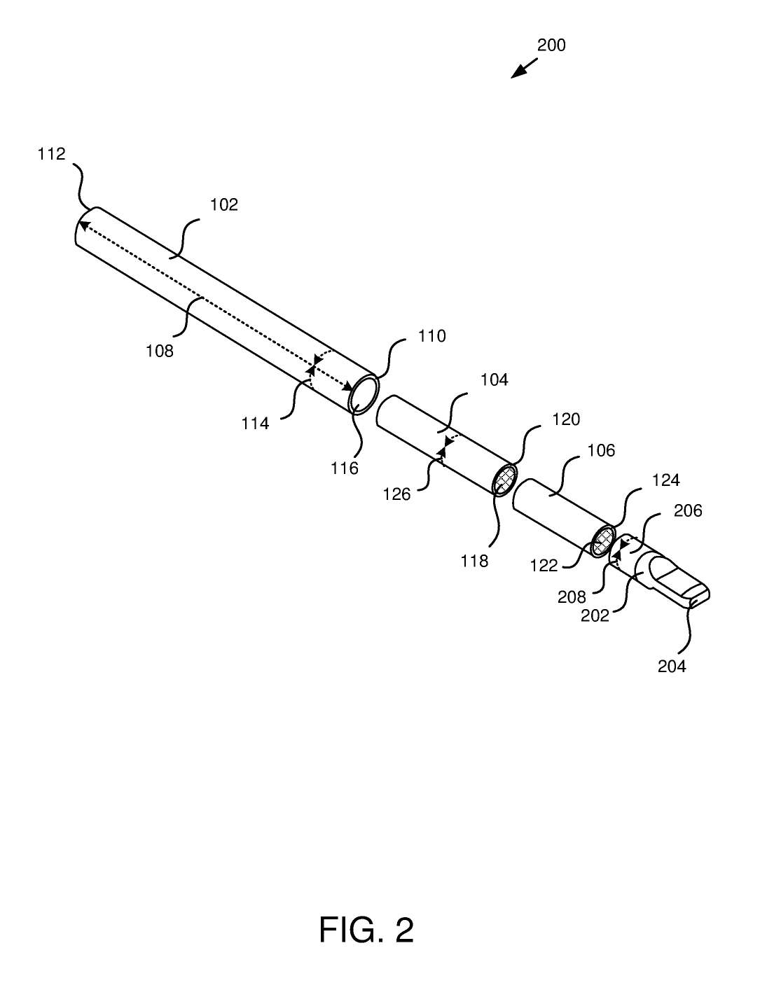

FIG. 2 is an exploded perspective view illustrating one embodiment of another apparatus 200 to inhale aromas in accordance with the present subject matter. In certain embodiments, the apparatus is substantially similar to the apparatus 100 discussed above with respect to FIG. 1. That is, in one embodiment, the apparatus 200 includes a cylindrical tube 102, an aroma dispensing member 104, and a filter 106. The cylindrical tube 102, the aroma dispensing member 104, and the filter 106 of apparatus 200 are substantially similar to and operate in a manner substantially similar to the cylindrical tube 102, the aroma dispensing member 104, and the filter 106 of apparatus 100.

In certain embodiments, the apparatus 200 also includes a mouthpiece 202. The mouthpiece 202 is coupleable to the proximal end 110 of the cylindrical tube 102. In one embodiment, one end of the mouthpiece 202 includes a cylindrical coupling flange 206. An outer diameter 208 of the cylindrical coupling flange 206 is slightly smaller than an inner diameter of the cylindrical tube 102 such that the cylindrical coupling flange 206 can be received within the interior 116 of the cylindrical tube 102. In certain embodiments, the difference in size between the outer diameter 208 of the cylindrical coupling flange 206 and the inner diameter of the cylindrical tube 102 is small enough that the cylindrical coupling flange 206 is press fit into the cylindrical tube 102.

To assemble the apparatus 200 the aroma dispensing member 104 is positioned within the interior 116 of the cylindrical tube 102 at a position adjacent the distal end 112 of the cylindrical tube. The filter 106 is then inserted into the interior 116 of the cylindrical tube 102 at a position near the proximal end 110 of the cylindrical tube 102. The cylindrical coupling flange 206 of the mouthpiece 202 is then inserted into the interior 116 of the cylindrical tube 102 at the proximal end 110 of the cylindrical tube 102 to couple the mouthpiece 202 to the cylindrical tube 102. In this manner, the mouthpiece 202 assists in keeping the aroma dispensing member 104 and the filter 106 positioned within the interior 116 of the cylindrical tube.

A passageway 204 extends through the mouthpiece 202. The passageway 204 leads from the interior 116 of the cylindrical tube 102 to an exterior of the mouthpiece 202. Thus, when a user inhales through the mouthpiece, air flows from the distal end 112 of the cylindrical tube 102, through the aroma dispensing member 104, through the filter 106, through the passageway 204 in the mouthpiece 202 and into the user's mouth.

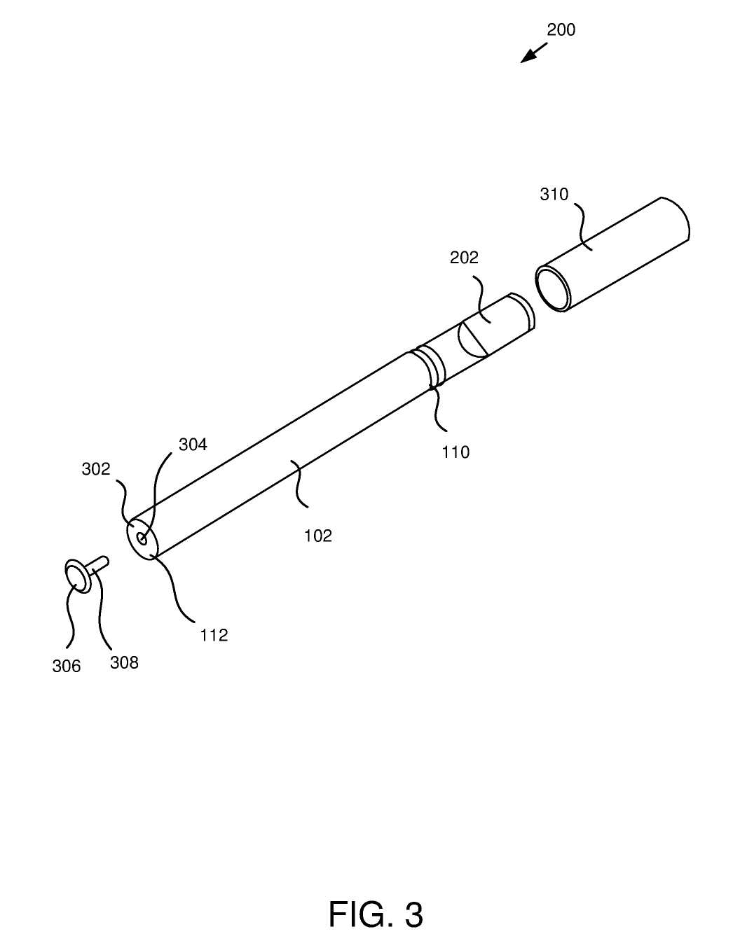

FIG. 3 is a perspective view illustrating one embodiment of the assembled apparatus 200 to inhale aromas of FIG. 2 in accordance with the present subject matter. In the embodiment illustrated in FIG. 3, the distal end 112 of the cylindrical tube 102 is clearly illustrated.

As discussed above, to assemble the apparatus 200, the aroma dispensing member 104 is positioned within the interior 116 of the cylindrical tube 102 at a position adjacent the distal end 112 of the cylindrical tube. The filter 106 is then inserted into the interior 116 of the cylindrical tube 102 at a position near the proximal end 110 of the cylindrical tube 102. The cylindrical coupling flange 206 of the mouthpiece 202 is then inserted into the interior 116 of the cylindrical tube 102 at the proximal end 110 of the cylindrical tube 102 to couple the mouthpiece 202 to the cylindrical tube 102.

In the embodiment illustrated in FIG. 3, the distal end 112 of the cylindrical tube 102 includes a barrier 302 that covers at least a portion of the distal end 112 of the cylindrical tube 102. The barrier 102 including at least one orifice 304 that extends from the interior 310 of the cylindrical tube 102 to the exterior of the cylindrical tube 102. As discussed above, the orifice 304 allows air to enter the interior 310 of the cylindrical tube 102 when the user inhales through the mouthpiece 202. The barrier 302 acts as a stopper to keep the aroma dispensing member 104 disposed within the interior 116 of the cylindrical tube 102. The mouthpiece 202 acts as a stopper at the proximal end 110 of the cylindrical tube 102 to keep the filter 106 positioned within the interior 116 of the cylindrical tube 102.

The apparatus 200, in certain embodiments, also includes a stopper 306. The stopper 306 includes a plug 308 positionable within the orifice 304 to limit fluid communication between the interior 116 of the cylindrical tube 102 and the exterior of the cylindrical tube 102 through the distal end 112 of the cylindrical tube 102. In other embodiments, a cap (not shown) may be used to cover the distal end 112 of the cylindrical tube 102. In either embodiment, the plug 308 or the cap limits the escape of aromas from within the interior 116 of the cylindrical tube 102 when the plug 308 or the cap are couple to the distal end 112 of the cylindrical tube 102.

In an exemplary embodiment, the apparatus 200 also includes a cover 310. The cover 310 is positionable about the mouth piece 202. The cover 310 limits fluid communication between the interior 116 of the cylindrical tube 102 and the exterior of the cylindrical tube 102 through the mouthpiece 202 (and therefore through the proximal end 110 of the cylindrical tube 102.) When the plug 308 or the cap are couple to the distal end 112 of the cylindrical tube 102 and the cover 310 is positioned on the mouth piece 202, the aroma contained within the aroma dispensing member 104 is maintained within the interior 116 of the cylindrical tube 102.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus to inhale aromas, the apparatus comprising:
a cylindrical tube elongated in a lengthwise direction, the cylindrical tube extending between a proximal end and a distal end, wherein the proximal end of the cylindrical tube is in fluid communication with an exterior of the cylindrical tube and wherein the distal end of the cylindrical tube is in fluid communication with an exterior of the cylindrical tube;
an aroma dispensing member positionable within an interior of the cylindrical tube between the proximal end of the cylindrical tube and the distal end of the cylindrical tube; and
a filter positionable within the interior of the cylindrical tube, the filter positionable between the aroma dispensing member and the proximal end of the cylindrical tube.

2. The apparatus of claim 1, wherein the distal end of the cylindrical tube includes a barrier, the barrier including at least one orifice extending from the interior of the cylindrical tube to the exterior of the cylindrical tube.

3. The apparatus of claim 2, further comprising a stopper, the stopper configured to limit fluid communication between the interior of the cylindrical tube and the exterior of the cylindrical tube through the distal end of the cylindrical tube.

4. The apparatus of claim 2, wherein the stopper comprises a plug positionable within the at least one orifice.

5. The apparatus of claim 2, wherein the stopper comprises a cap positionable about the distal end of the cylindrical tube.

6. The apparatus of claim 1, further comprising a mouthpiece coupleable to the proximal end of the cylindrical tube, the mouthpiece comprising a passageway leading from interior of the cylindrical tube to the exterior of the cylindrical tube.

7. The apparatus of claim 6, further comprising a cover positionable about the mouth piece, the cover configured to limit fluid communication between the interior of the cylindrical tube and the exterior of the cylindrical tube through the proximal end of the cylindrical tube.

8. The apparatus of claim 1, further comprising an aroma dispensing substance disposed within the aroma dispensing member, the aroma dispensing substance having at least one therapeutic property.

9. The apparatus of claim 8, wherein the aroma dispensing substance is refillable.

10. The apparatus of claim 8, wherein the at least one therapeutic property comprises a property selected from an appetite suppressing agent and a calming agent.

11. The apparatus of claim 8, wherein the aroma dispensing substance comprises an essential oil.

12. The apparatus of claim 1, wherein the aroma dispensing member comprises a matrix of polyester fibers.

13. The apparatus of claim 12, wherein the matrix of polyester fibers is disposed within a pliable sheath, the pliable sheath having an outer diameter smaller than an inner diameter of the cylindrical tube.

14. The apparatus of claim 1, wherein the filter comprises a matrix of polyester fibers.

15. The apparatus of claim 14, wherein the matrix of polyester fibers is disposed within a pliable sheath, the pliable sheath having an outer diameter smaller than an inner diameter of the cylindrical tube.

16. An aroma inhaler comprising:
a cylindrical tube elongated in a lengthwise direction, the cylindrical tube extending between a proximal end and a distal end, wherein the proximal end of the cylindrical tube is in fluid communication with an exterior of the cylindrical tube and wherein the distal end of the cylindrical tube is in fluid communication with an exterior of the cylindrical tube;
an aroma dispensing member positionable within an interior of the cylindrical tube between the proximal end of the cylindrical tube and the distal end of the cylindrical tube;
an aroma dispensing substance disposed within the aroma dispensing member, the aroma dispensing substance having at least one therapeutic property; and
a filter positionable within the interior of the cylindrical tube, the filter positionable between the aroma dispensing member and the proximal end of the cylindrical tube.

17. The aroma inhaler of claim 16, wherein the at least one therapeutic property comprises a property selected from an appetite suppressing agent and a calming agent.

18. The aroma inhaler of claim 16, wherein the aroma dispensing substance comprises an essential oil.

19. The aroma inhaler of claim 16, further comprising;
a stopper configured to limit fluid communication between the interior of the cylindrical tube and the exterior of the cylindrical tube through the distal end of the cylindrical tube;
a mouthpiece coupleable to the proximal end of the cylindrical tube, the mouthpiece comprising a passageway leading from interior of the cylindrical tube to the exterior of the cylindrical tube; and
a cover positionable about the mouth piece, the cover configured to limit fluid communication between the interior of the cylindrical tube and the exterior of the cylindrical tube through the proximal end of the cylindrical tube.

20. An apparatus to inhale aromas, the apparatus comprising:
a cylindrical tube elongated in a lengthwise direction, the cylindrical tube extending between a proximal end and a distal end, wherein the proximal end of the cylindrical tube is in fluid communication with an exterior of the cylindrical tube and wherein the distal end of the cylindrical tube is in fluid communication with an exterior of the cylindrical tube;
an aroma dispensing member positionable within an interior of the cylindrical tube between the proximal end of the cylindrical tube and the distal end of the cylindrical tube;
a filter positionable within the interior of the cylindrical tube, the filter positionable between the aroma dispensing member and the proximal end of the cylindrical tube;
a stopper configured to limit fluid communication between the interior of the cylindrical tube and the exterior of the cylindrical tube through the distal end of the cylindrical tube;
a mouthpiece coupleable to the proximal end of the cylindrical tube, the mouthpiece comprising a passageway leading from interior of the cylindrical tube to the exterior of the cylindrical tube; and
a cover positionable about the mouth piece, the cover configured to limit fluid communication between the interior of the cylindrical tube and the exterior of the cylindrical tube through the proximal end of the cylindrical tube.
